(19) United States
(12) Patent Application Publication
(10) Pub. No.: US 2005/0267470 A1
(43) Pub. Date: Dec. 1, 2005

(54) Spinal stabilization system to flexibly connect vertebrae

(76) Inventor: Duncan Q. McBride, Rolling Hills, CA (US)

Correspondence Address: Brian M. Berliner;O'MELVENY & MYERS LLP, 400 South Hope Street, Los Angeles, CA, 90071-2899, US

(21) Appl. No.: 10/846,400

(22) Filed: May 13, 2004

(51) Int. Cl. A61B 17/58

(52) U.S. Cl. 606/61

(57) ABSTRACT

A surgically implanted spinal stabilization system uses posterior anchor hooks attached to vertebrae to retain elastic bands to retain flexibility and mobility while maintaining alignment and preventing excessive motion and deformity. The elastic bands may parallel the longitudinal axis of the spine, or, for enhanced promotion of alignment, they may also arranged in a diagonally crossing configuration. Multi-level fixation can be achieved using the spinal stabilization system with longer elastic bands. A method of applying the spinal stabilization system using an elastic band application tool facilitates simple, rapid application of the system to a patient.

### BACKGROUND OF THE INVENTION

1. Field of the Invention

The present invention relates to devices used to provide spinal stabilization and more particularly to systems for spinal stabilization allowing flexibility of the vertebrae.

2. Description of Related Art

The human spine is comprised of 33 stacked vertebrae extending from the base of the skull to the tailbone with cartilaginous disks sandwiched between each two adjacent vertebrae providing a cushion and easing movement of the vertebrae relative to each other. In a healthy spine, this interconnected arrangement of vertebrae and disks supports loads while remaining highly flexible—since each vertebra can move with respect to the adjoining vertebrae, the spine can bend and twist to a remarkable degree. However, with a spinal injury, deformity or degeneration, even at a single disk level, the spine's ability to support load can be greatly compromised. As a result, a person's spinal injury often leads to great discomfort when standing and an inability to lift heavy objects.

While spinal injuries such as herniated disks are currently treatable, certain treatments have undesirable results. Traditionally, incompetent disks have been surgically treated by solidly fusing the vertebrae adjacent to the injured disk or disks. In this method, two or more vertebrae are fused with bone grafts and internal devices such as cages or metal screws and rods to heal into a single solid bone. This traditional spinal fusion method is also used in certain instances to treat injuries to vertebrae, abnormal curvatures of the spine (scoliosis or kyphosis), and weak or unstable spine caused by degenerative changes, infections or tumors. While this traditional method of treatment for spinal injuries can restore the strength of the spinal column and its proper curvature, the fusion of adjacent vertebrae restores strength at the expense of flexibility. Therefore, a person who has undergone traditional vertebral fusion surgery will lose a degree of bending and twisting flexibility in the spine. Also, the disks adjacent to the fused levels degenerate at an increased rate, often requiring extension of the fusion. Furthermore the traditional fusion treatment (and its accompanying lack of flexibility) is essentially irreversible. Pseudoarthrosis (or failed fusion) also is a risk of all attempts at achieving solid bony fusion, usually requiring reoperation.

Certain spinal conditions may benefit from surgical stabilization to maintain posterior curvature (lordosis) and alignment. This is particularly true after posterior decompressive surgery procedures that remove bone, ligaments, joints and disks to relieve pressure on nervous tissues. Such procedures can weaken the spinal structure and result in post-operative increase in misalignments or reversal of normal lordosis. Fusion would not be required in these situations if a posterior, flexible device that could preserve normal spinal alignment was implanted. This would be an advantage over fusion because more normal motion would be preserved, no boney fusion growth would be required, and adjacent level integrity would not be threatened.

Others have attempted to address the shortcomings of the traditional spinal fusion method, however, these attempts have had limited success and introduced further shortcomings. Several prior art systems employ a complex array of rods, springs and posts to position the spinal column of a wearer as desired. In these systems, two rods parallel to the desired axial configuration of the spine are attached to the spine with posts attached to each vertebra. These devices are designed to be removable and allow some degree of flexibility while maintaining the proper alignment and support of the spine. However, these devices are complex, involving a large number of component parts. This complexity would undesirably lead to long application and removal times and the need for extensive training by the applying surgeon. Furthermore, the rigid alignment rods and other hardware would negatively impact flexibility (though not as much as the traditional vertebral fusion method).

Other devices attempting to address the shortcomings of the traditional spinal fusion method have done so by joining vertebrae together with cables or dampers attached to pairs of posts attached to individual vertebrae. In these prior art devices, the cables or dampers may run between vertebrae along the axis of the spine or they may run in a crossing pattern between vertebrae. In some of the cable-based spinal stabilization devices, dampening devices have been substituted for the cables running between vertebrae parallel to the axis of the spine. These prior art devices address the traditional vertebral fusion's removability shortcoming but do not address the flexibility shortcomings. Tension in the cables used in these devices provides compression across the disk space. Therefore, these devices restrict the wearer's range of mobility in bending and flexure. Moreover, since the cables used in these devices are much less elastic than the cartilage, ligaments, and other soft tissues that define mobility in a healthy spine, these devices create an unnatural firm stop at the limits of movement

Therefore, there is a need for a spinal stabilization device that is simple, facilitating ease of application, permits the wearer to retain nearly a full range of mobility and flexibility in the spine, and is removable.

### SUMMARY OF THE INVENTION

The present invention addresses the shortcomings of the prior art, by providing a system for posterior spinal stabilization that is simple, permits the user to retain a large range of spinal flexibility and mobility while preventing excessive motion and maintaining proper alignment, and is removable. The system is also universally applicable to all levels of the human spine.

The spinal stabilizer of the present invention is a posterior spinal implant system comprising at least one elastic band retained by at least one pair of anchor hooks. The small number of component elements in the spinal stabilization system of the present invention facilitates relatively quick surgical application and removal times. The flexible nature of the elastic bands allows flexibility and mobility of the wearer's spine while simultaneously maintaining alignment and preventing excessive motion and deformity

The anchor hooks of the present invention are of a material, such as titanium, that is strong, durable, and can be safely surgically implanted. The anchor hooks of the present invention are to be screwed into pilot holes drilled in locations appropriate to the level of the vertebra to be flexibly connected. The screw locations will preferably be in the lateral masses for cervical vertebrae and in the pedicles in lumbar and thoracic vertebrae. Varying sizes of screw threads and anchor hooks may be used in the system of the present invention to facilitate application on different sized vertebrae along the length of the spine.

The spinal stabilization system of the present invention comprises three different types of anchor hooks: eye hooks and double hooks and multiple hooks. Eye hooks comprise a crimpable hook section connected to the head of a screw thread. The upper surface of the crimpable hook section has a groove in it to mate in the correct alignment with an elastic band application tool. Eye hooks are oriented so that the open end of the crimpable hook section faces away from the center of fixation. Double hooks comprise two crimpable hook sections connected to the head of a screw thread. Multi-hooks will be able to crimp over three bands oriented in different directions when crisscross banding is performed. Depending on the affected vertebrae and the desired treatment, eye hooks may be used alone to flexibly connect two adjacent vertebrae or in conjunction with double hooks for multi-level fixation. Multi-level fixation may be used to prevent post laminectomy kyphosis and maintain decompressive lordosis. All hooks are configured to be crimped around the elastic bands. The crimpable hook sections hooks feature a recess at the end of the crimpable hook section adjacent to the head of the screw thread and a tapered tip at the opposite end of the crimpable hook section. The recess facilitates application and retention of the elastic band to the hook shaped portion. The tapered tip on the elastic band retaining portion allows for flush closure when the elastic band retaining portions are crimped around an elastic band, preventing release of the elastic band.

By combining different sizes and resistances of elastic bands with different sizes and types of anchor hooks, multiple embodiments of the present invention can be made. For example, a first embodiment representing treatment for a simple case in which two adjacent vertebrae are to be flexibly connected, two pairs of eye-hooks (one pair per vertebra) would be screwed into pilot holes drilled into the appropriate locations on the vertebrae. The first embodiment further comprises a pair of elastic bands with the desired length and resistance properties. Each of the elastic bands parallels the longitudinal axis of the spine and connects an eye-hook on one vertebra with the corresponding eye-hook on the other vertebra. A second, slightly more complex embodiment could be used to flexibly connect two adjacent vertebrae where enhanced promotion of alignment is desired. The second embodiment of the invention comprises all of the elements of the first embodiment of the invention arranged as in the first embodiment of the invention, and further comprises a second pair of elastic bands, with the desired length and resistance for diagonal use, arranged in a crossing diagonal pattern between the anchor hooks in the vertebrae (i.e. one elastic band of the second pair would be retained by the upper left anchor and the lower right anchor and the second elastic band of the second pair would be retained by the upper right anchor and the lower left anchor).

Additional embodiments of the present invention could provide stabilization to more than two vertebrae. For example, a third embodiment of the invention could provide multilevel flexible connection of the spine by flexibly attaching three or more vertebrae. The third embodiment comprises two pairs of eye hooks, one pair for each of the upper and lower vertebrae to be flexibly connected plus one pair of double hooks for each intermediate vertebra to be flexibly connected. A pair of elastic bands of the desired length parallels the longitudinal axis of the spine and is retained by the anchor hooks. One of the elastic bands connect all of the anchor hooks on the left side (in relation to the longitudinal axis of the spine) of the spinal column, and the other of the pair of elastic bands would connect all of the anchor hooks on the right side (in relation to the longitudinal axis of the spine) of the spinal column. A fourth embodiment of the invention could provide multi-level fixation with enhanced promotion of alignment. This fourth embodiment combines the multilevel stabilization arrangement of the third embodiment with additional pairs of elastic bands, arranged in a crossing diagonal pattern between adjacent vertebrae as in the second embodiment. These four embodiments provide examples of several of the spinal stabilization arrangements possible within the scope of the present invention. However, it should be recognized that many other combinations of the components of the present invention, may be made. While not individually listed, these combinations are within the spirit and scope of the present invention.

The elastic bands of the spinal stabilization system of the present invention are composed of a material that allows flexibility to a limit while being able to withstand millions of contractions with no significant degradation in flexibility. The material of the elastic bands must also be safe for implanting into humans and resist degradation. The preferred material for the elastic band is reinforced silastic, although other materials with the described properties are also considered within the scope of the present invention. Different thicknesses of elastic bands, with corresponding differences in resistance to extension may be used in the system of the present invention. Therefore, the system of the present invention is adaptable to provide varying degrees of mobility and flexibility depending on the desired treatment. The elastic bands may be color coded by resistance to facilitate application of the desired resistance level by the applying physician

Several lengths of elastic bands may be employed in the spinal stabilization system of the present invention. The various lengths of elastic bands allow the spinal stabilization system to be applied at any desired location along the length of a wearer's spine: shorter bands would be used on cervical vertebrae, and progressively longer sized bands would be used on lower vertebrae in the thoracic and lumbar regions. Still longer elastic bands would be used in the system of the present invention to accomplish multi-level fixation. In multi-level fixation, more than two vertebrae would be flexibly connected by the system of the present invention with a pair of eye hooks anchored into the upper vertebra to be flexibly connected, a pair of double hooks anchored in each of the intermediate vertebrae to be flexibly connected, and a pair of eye hooks anchored into the lower vertebra to be flexibly connected. The elastic bands of the present invention will further comprise a continuous radio opaque stripe. This radio opaque stripe would allow the elastic bands of the present invention to be monitored by x-ray. A breakage of the elastic band would be visible as a discontinuity in the radio opaque stripe as viewed on an x-ray image. Likewise, the position of the elastic bands relative to the anchor hooks could be monitored with x-ray imaging.

The spinal stabilization system of the present invention is applied by screwing pairs of anchor hooks into corresponding pairs of pilot holes drilled in vertebrae. An elastic band application tool may then be used to stretch an elastic band running parallel to the axis of the spine, over anchor hooks in the upper and lower vertebrae to be flexibly connected. The elastic band application tool comprises two lever arms, a locking mechanism to hold the elastic band open the desired amount, an anchor hook interface to mate with grooves in the anchor hooks in tongue-in-groove fashion, and an elastic band rolling mechanism that slides the elastic band over the anchor hooks. Once the elastic band is properly positioned over the anchor hooks, a crimping tool is used to close the anchor hooks over the elastic band. If the spinal stabilization is a multilevel fixation, the elastic band is applied and secured, as described above, to the eye hooks in the upper and lower vertebrae to be flexibly connected. Then the elastic band is rolled over the double hooks in the intermediate vertebrae to be flexibly connected with the elastic band application tools. The double hooks are then crimped over the elastic band with a crimping tool.

### BRIEF DESCRIPTION OF THE DRAWINGS

FIG. 1 is a diagram showing a first embodiment of the spinal stabilization system of the present invention as applied to two vertebrae.

FIG. 2 is a diagram showing a second embodiment of the spinal stabilization system of the present invention as applied to two vertebrae.

FIG. 3 is diagram showing a third embodiment of the spinal stabilization system of the present invention as applied to several vertebrae.

FIG. 4 is a diagram showing a fourth embodiment of the spinal stabilization system of the present invention as applied to several vertebrae.

FIG. 5 is a side view diagram showing anchor hooks of the present invention.

FIG. 6 is a side view diagram showing elastic bands of the present invention.

FIGS. 7A and 7B are diagrams showing application of an elastic band to anchor hooks using the method of the present invention.

### DETAILED DESCRIPTION OF THE INVENTION

The present invention provides a spinal stabilization system that overcomes the limitations of prior-art spinal stabilization systems. In the detailed description that follows, like element numerals are used to indicate like elements that appear in one or more of the drawings.

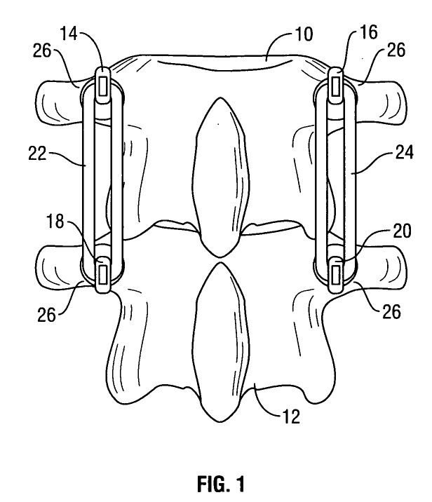

FIG. 1 depicts a first embodiment of the system of the present invention. In the first embodiment, the spinal stabilization system of the present invention is a system for flexibly connecting an upper vertebra 10 and an adjacent lower vertebra 12. The system of the first embodiment comprises: a left anchor hook 14 and a right anchor hook 16 attachable to the upper vertebra 10, a left anchor hook 18 and a right anchor hook 20 attachable to the lower vertebra 12, a first elastic band 22 retained by the left anchor hook 14 in the upper vertebra 10 and the left anchor hook 18 in the lower vertebra 12 and substantially parallel to the longitudinal axis of the spine, and a second elastic band 24 retained by the right anchor hook 16 in the upper vertebra 10 and the right anchor 20 hook in the lower vertebra 12 and substantially parallel to the longitudinal axis of the spine.

As is evident from the first embodiment in FIG. 1, the system of the present invention flexibly attaches two vertebrae 10, 12 while allowing the vertebrae to move relative to each other to the extent of the elasticity of the elastic bands 22, 24, thereby allowing the wearer to have a high degree of mobility and flexibility. Further, the few component parts of the present invention facilitate ease of application. FIG. 1 depicts the system of the present device as applied to two lumbar vertebrae 10, 12, with the anchor hooks 14, 16, 18, 20 attached to the vertebrae 10, 12 at the pedicle location 26. However, it should be recognized that the system of the present invention may be applied to any two adjacent vertebrae along the length of the spine. The location of the anchor hooks, however would vary from lateral masses in the cervical vertebrae to pedicles 26 in the thoracic and lumbar vertebrae.

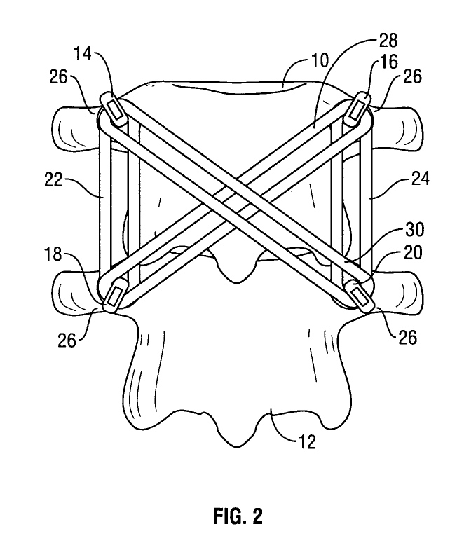

A second embodiment of the present invention is depicted in FIG. 2. The second embodiment flexibly connects two vertebrae 10, 12 while providing enhanced promotion of alignment and preventing listhesis. The second embodiment comprises all of the elements of the first embodiment, as depicted in FIG. 1, and further comprises: a third elastic band 28 retained by the left anchor hook 14 in the upper vertebra 10 and the right anchor hook 20 in the lower vertebra 12; and a fourth elastic band 30 retained by the right anchor hook 16 in the upper vertebra 10 and the left anchor hook 18 in the lower vertebra 12.

The second embodiment of the present invention allows flexibility and mobility while enhancing promotion of alignment. Further, the second embodiment of the present invention has very few component elements as compared with complex rod-based systems of the prior art. As with the first embodiment of the invention, the second embodiment may be applied, by varying attachment locations for the anchor hooks, to any two adjacent vertebrae along the length of the spine.

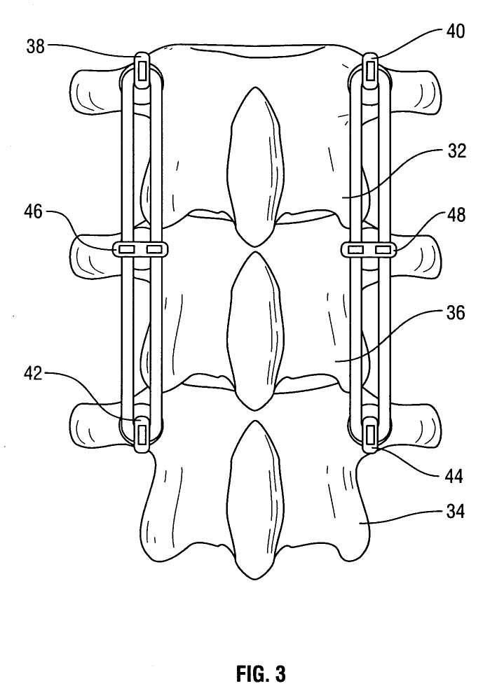

A third embodiment of the present invention is depicted in FIG. 3. The third embodiment of the present invention allows flexible connection of more than two vertebrae 32, 34, 36. The third embodiment of the present invention comprises: a left anchor hook 38 and a right anchor hook 40 attachable to an upper vertebra 32; a left anchor hook 42 and a right anchor hook 44 attachable to a lower vertebra 34; a left anchor hook 46 and a right anchor hook 48 attachable to each of the at least one intermediate vertebrae 36; a first elastic band 50 retained by the left anchor hook 38 in the upper vertebra 32, the left anchor hooks 46 in the at least one intermediate vertebrae 36, and the left anchor hook 42 in the lower vertebra 34, wherein the first elastic band 50 is substantially parallel to the longitudinal axis of the spine; and a second elastic band 52 retained by the right anchor hook 40 in the upper vertebra 32, the right anchor hooks 48 in the at least one intermediate vertebrae 36, and the right anchor hook 44 in the lower vertebra 34, wherein the second elastic band 52 is substantially parallel to the longitudinal axis of the spine.

When multi-level fixation is desired, the third embodiment of the present invention allows the user to retain mobility and flexibility while providing support to the spine. The third embodiment of the present invention has fewer component elements than complex prior art rod-based devices, thereby facilitating ease of application and removal.

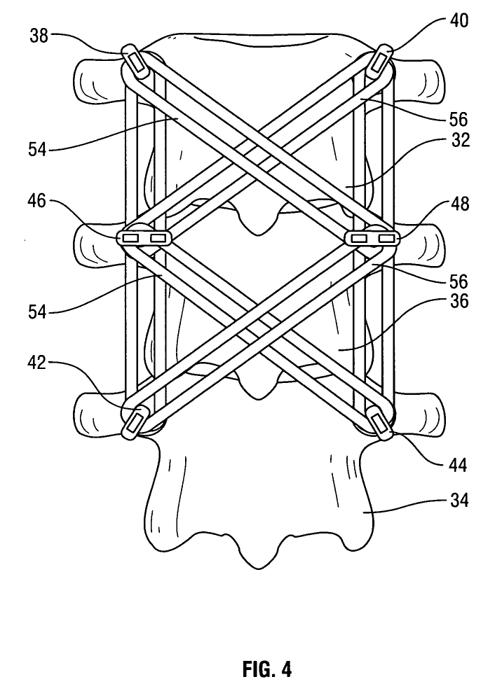

A fourth embodiment of the present invention is depicted in FIG. 4. The fourth embodiment of the present invention flexibly attaches more than two vertebrae 34, 36, 38 while enhancing promotion of alignment. The fourth embodiment comprises all of the elements of the third embodiment, as depicted in FIG. 3, and further comprises: at least one pair of diagonal crossing elastic bands 54, 56 wherein one 54 of each of the at least one pair of diagonal crossing elastic bands is retained by the right anchor hook 44 in a flexibly connected vertebra 34 and the left anchor hook 46 in an adjacent flexibly connected vertebra 36 and the other 56 of each of the at least one pair of diagonal crossing elastic bands is retained by the left anchor hook 42 in the flexibly connected vertebra 34 and the right anchor hook 48 in the adjacent flexibly connected vertebra 36.

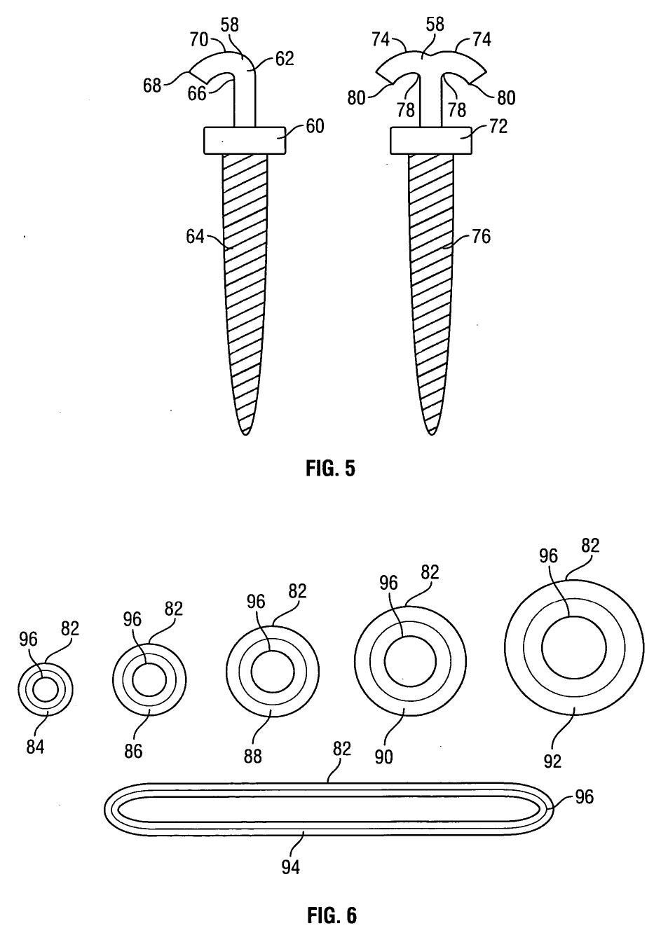

FIG. 5 depicts two types of anchor hooks 58 used in the system of the present invention. Each anchor hook 58 attached to the upper and lower vertebrae to be flexibly connected in each of the embodiments described above is an eye hook 60. Each eye hook 60 comprises: a crimpable hook section 62 affixed to a screw thread 64, and wherein the crimpable hook section 62 further comprises a recess 66 where the crimpable hook section meets the screw thread and a tapered tip 68 opposite where the crimpable hook section meets the screw thread. The recess 66 in the eye hook 60 facilitates application of an elastic band to the eye hook 60. Once an elastic band, (or in embodiments of the system with enhanced promotion of alignment, more than one elastic band) is applied to the eye hook 60, a crimping tool is used to close the crimpable hook section 62 over the elastic band. The tapered tip 68 allows the crimpable hook section 62 to be crimped flushly around the elastic band. The eye hooks 60 are to be attached to the individual vertebrae such that the end of the crimpable hook section 62 with the tapered tip 68 faces away from the center of fixation (i.e. the tapered tip 68 will be facing up for eye hooks 60 attached to an upper vertebra and facing down for eye hooks attached to a lower vertebra). For ease of applying elastic bands to the eye hooks 60, the eye hooks 60 preferably further comprise a groove 70 in the crimpable hook section 62 configured to interface with an elastic band application tool.

The anchor hooks 58 used in intermediate vertebrae in embodiments of the present invention providing multi-level fixation are double hooks 72. The double hooks each comprise two crimpable hook sections 74 affixed to a screw thread 76. The crimpable hook sections 74 of the double hooks 72 each further comprise a recess 78 where the crimpable hook section 74 meets the screw thread 76 and a tapered tip 80 opposite where the crimpable hook section 74 meets the screw thread 76. The recess 78 in each crimpable hook section 74 of the double hook 72 facilitates application of an elastic band to the double hook 72. Once an elastic band (or in embodiments of the system with enhanced promotion of alignment, more than one elastic band) is applied to each crimpable hook section 74 of the double hook 72, a crimping tool is used to close the crimpable hook sections 74 over the band. The tapered tip 80 allows each crimpable hook section 74 to be crimped flushly around the elastic band. The double hooks 72 are to be attached to the individual vertebrae oriented such that the crimpable hook sections 74 would open perpendicularly to the longitudinal axis of the spine, thereby allowing elastic bands running parallel to the longitudinal axis of the spine to be easily retained by the crimpable hook sections 74. Although not depicted, another type of anchor hook, a multi-hook, may be used where the embodiment of the spinal stabilization system of the present invention results in three bands oriented in different directions being retained by the same anchor hook.

The anchor hooks 58 must be composed of a material that is strong, durable, and capable of being implanted into humans without adverse reaction. The anchor hooks 58 of the present invention are preferably composed of titanium. Multiple sizes of anchor hooks 58 are contemplated within the scope of the present invention. The treating physician or surgeon can select an anchor hook 58 of a size appropriate to the vertebra to which it will be attached. Therefore, through the use of multiple sizes of anchor hooks 58, the system of the present invention is adaptable to flexibly connect the various sizes of vertebrae along the length of a spine. Likewise, the system of the present invention is adaptable to being applied to varying sizes of vertebrae in spines of people of different ages and builds.

FIG. 6 depicts the elastic bands 82 to be used in the system of the present invention. The elastic bands 82 of the present invention are composed of a material that allows flexibility and is capable of withstanding millions of extension and contraction cycles. The material of the elastic bands 82 must also be capable of being implanted into humans with little chance of an adverse reaction. The elastic bands 82 of the present invention are preferably composed of reinforced silastic. Elastic bands 82 of varying lengths 84, 86, 88, 90, 92, 94 and resistances are contemplated within the scope of the present invention. Preferably, the elastic bands used in a particular application will be of a length corresponding to a desired distance between the anchor hooks that retain the band. Different lengths of elastic bands 82 will correspond to their intended area of use in the spine. For example, elastic bands for use in the cervical region 84, 86 will be shortest, and elastic bands for use in the thoracic 88, 90 and lumbar 92 regions will be progressively longer. Still longer elastic bands 94 are used in multi-level fixation. The thickness of an elastic band 82 will determine its resistance to extension. Multiple thicknesses and therefore resistances of elastic bands 82 may be chosen for each length of elastic band 82 depending on the desired resistance to extension for the treatment chosen. Alternatively, bands could be with internal variations in resistance while maintaining uniform thickness. Preferably the elastic bands 82 are color-coded by resistance to facilitate selection of the elastic band 82 with the desired flexion capability. Therefore, the system of the present invention is adaptable to meet differing flexibility and mobility needs. The elastic bands 82 may further comprise a continuous radio opaque stripe 96. The radio opaque stripe 96 allows for a diagnostic review of the elastic bands 82 with X-ray imaging. Such a review could detect breakage or improper application of the elastic bands 82.

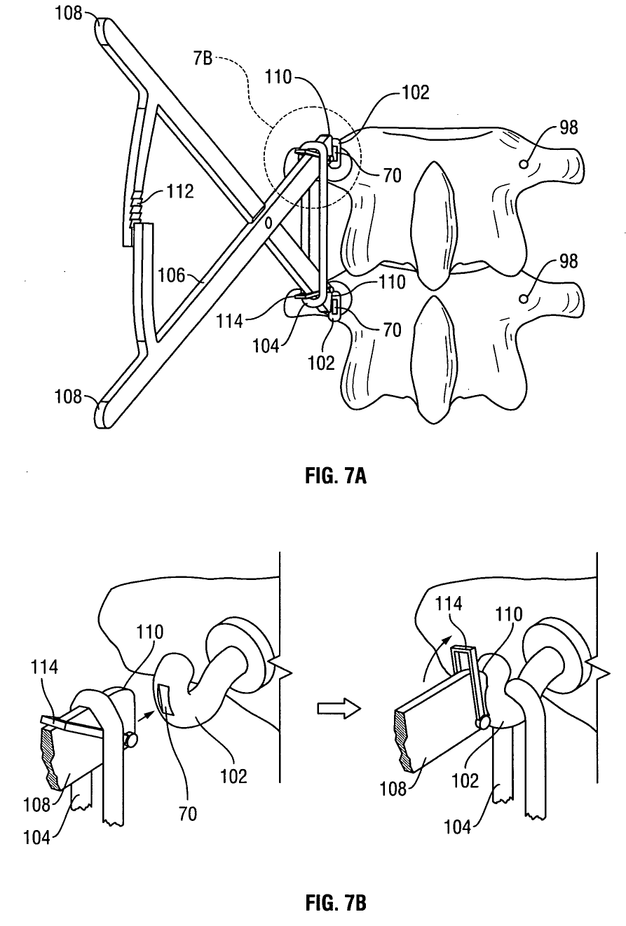

FIG. 7A depicts application of the spinal stabilization system through a novel method of the present invention. The method of applying the system of the present invention comprises the steps of: drilling two pilot holes 98 in each vertebra 100 to be flexibly attached; applying an anchor hook 102 to each pilot hole 98 in the vertebrae 100 to be flexibly attached; applying elastic bands 104 to the anchor hooks 102; and crimping the anchor hooks 102 to retain the elastic bands 104. Pilot holes 98 are drilled in the desired location on the vertebrae 100 to be flexibly attached. The pilot hole 98 location may vary depending on the region of the spine to be flexibly attached. Preferably pilot holes 98 will be drilled in lateral masses in cervical vertebrae and in pedicles in lumbar and thoracic vertebrae. Anchor hooks 102 are then attached to the vertebrae 100 by screwing each anchor hook 102 into a pilot hole 98. Elastic bands 104 are then applied to the anchor hooks 102 as appropriate for the desired fixation. Preferably, an elastic band application tool 106 would be used to apply the elastic bands 104 to the desired anchor hooks 102. The elastic band application tool 106 comprises two lever arms 108, an anchor hook interface 110, a locking mechanism 112, and an elastic band rolling mechanism 114. An elastic band 104 can be picked up and extended to the desired length with the elastic band application tool 106. The locking mechanism 112 on the elastic band application tool 106, here depicted as a ratchet lock, then maintains the proper extension of the elastic band 104. The elastic band application tool 106 is then mated with the anchor hooks 102 to which the elastic band 104 will be applied. The anchor hook interface 110 on the elastic band application tool 106 mates with the groove 70 in each of the anchor hooks 102 in tongue-in-groove fashion. This mating is shown in closer detail in FIG. 7B. This mating allows proper positioning of the elastic band 104 to facilitate its application. The elastic band rolling mechanism 114, here depicted as a lever that slides the elastic band 104 onto the anchor hooks 102, then rolls the elastic band 104 off of the elastic band application tool 106 and onto the anchor hooks 102. Once the elastic band 104 has been applied to the anchor hooks 102, the anchor hooks 102 are crimped closed around the elastic band 104. The method of the present invention can be used to apply elastic bands 104 to eye hooks and double hooks.

Having thus described several embodiments of the spinal stabilization system, it should be apparent to those skilled in the art that certain advantages of the device have been achieved. It should also be appreciated that various modifications, adaptations, and alternative embodiments thereof may be made within the scope and spirit of the present invention. The invention is further defined by the following claims.

1. A system for flexibly connecting at least two vertebrae comprising:
at least one pair of anchor hooks attachable to the at least two vertebrae to be flexibly connected; and
at least one elastic band retained by the at least one pair of anchor hooks.

2. The system of claim 1, wherein:
the at least two vertebrae to be flexibly connected comprise:
an upper vertebra; and
an adjacent lower vertebra of a spine having a longitudinal axis;
the at least one pair of anchor hooks comprise:
a first left anchor hook and a first right anchor hook attachable to the upper vertebra; and
a second left anchor hook and a second right anchor hook attachable to the lower vertebra; and
the at least one elastic band comprises:
a first elastic band retained by the first left anchor hook in the upper vertebra and the second left anchor hook in the lower vertebra, wherein the first elastic band is substantially parallel to the longitudinal axis of the spine; and
a second elastic band retained by the first right anchor hook in the upper vertebra and the second right anchor hook in the lower vertebra, wherein the second elastic band is substantially parallel to the longitudinal axis of the spine.

3. The system of claim 2, further comprising:
a third elastic band retained by the first left anchor hook in the upper vertebra and the second right anchor hook in the lower vertebra; and
a fourth elastic band retained by the first right anchor hook in the upper vertebra and the second left anchor hook in the lower vertebra.

4. The system of claim 2, wherein the first and second anchor hooks attachable to the upper and lower vertebrae each comprises a crimpable hook section affixed to a screw thread, and wherein the crimpable hook section further comprises a recess where the crimpable hook section meets the screw thread and a tapered tip opposite where the crimpable hook section meets the screw thread.

5. The system of claim 4, wherein the first and second anchor hooks attachable to the upper and lower vertebrae further comprise a groove in the crimpable hook section configured to interface with an elastic band application tool.

6. The system of claim 2, wherein the elastic bands further comprise a continuous radio opaque stripe.

7. The system of claim 2, wherein the elastic bands are comprised of reinforced silastic material.

8. The system of claim 2, wherein the elastic bands are of a length corresponding to a region of the spine in which the upper and lower vertebrae are located.

9. The system of claim 2, wherein the elastic bands have a resistance to extension corresponding to a desired flexion capacity of the upper and lower vertebrae.

10. The system of claim 9, wherein the elastic bands have a thickness proportional to the resistance to extension.

11. The system of claim 9, wherein the resistance to extension of the elastic bands is defined by an internal variation in resistance.

12. The system of claim 9, wherein the elastic bands further comprise color coding representing the resistance to extension.

13. The system of claim 2, wherein the first and second anchor hooks are comprised of titanium material.

14. The system of claim 2, wherein each anchor hook is of a size appropriate to the vertebra to which it is attached.

15. A system for flexibly connecting an upper vertebra, a lower vertebra, and at least one intermediate vertebrae of a spine having a longitudinal axis comprising:
a first left anchor hook and a first right anchor hook attachable to the upper vertebra;
a second left anchor hook and a second right anchor hook attachable to the lower vertebra;
a third left anchor hook and a third right anchor hook attachable to each of the at least one intermediate vertebrae;
a first elastic band retained by the first left anchor hook in the upper vertebra, the third left anchor hooks in the at least one intermediate vertebrae, and the second left anchor hook in the lower vertebra, wherein the first elastic band is substantially parallel to the longitudinal axis of the spine; and
a second elastic band retained by the first right anchor hook in the upper vertebra, the third right anchor hooks in the at least one intermediate vertebrae, and the second right anchor hook in the lower vertebra, wherein the second elastic band is substantially parallel to the longitudinal axis of the spine.

16. The system of claim 15, wherein the anchor hooks attachable to the upper and lower vertebrae each comprise a crimpable hook section affixed to a screw thread, and wherein the anchor hooks attachable to each of the at least one intermediate vertebrae each comprise two crimpable hook sections affixed to a screw thread, and wherein the crimpable hook sections of the anchor hooks for upper, lower, and intermediate vertebrae each further comprise a recess where the crimpable hook section meets the screw thread and a tapered tip opposite where the crimpable hook section meets the screw thread.

17. The system of claim 15, further comprising at least one pair of diagonal crossing elastic bands wherein one of each of the at least one pair of diagonal crossing elastic bands is retained by the right anchor hook in a flexibly connected vertebra and the left hook in an adjacent flexibly connected vertebra and the other of each of the at least one pair of diagonal crossing elastic bands is retained by the left anchor hook in said flexibly connected vertebra and the right anchor hook in said adjacent flexibly connected vertebra.

18. The system of claim 17, wherein the anchor hooks attachable to the upper and lower vertebrae each comprise a crimpable hook section affixed to a screw thread, and wherein the anchor hooks attachable to each of the at least one intermediate vertebrae each comprise at least two crimpable hook sections affixed to a screw thread, and wherein the crimpable hook sections of the anchor hooks for upper, lower, and intermediate vertebrae each further comprise a recess where the crimpable hook section meets the screw thread and a tapered tip opposite where the crimpable hook section meets the screw thread.

19. The system of claim 15, wherein the anchor hooks attachable to the upper, lower, and intermediate vertebrae further comprise a groove in the crimpable hook section configured to interface with an elastic band application tool.

20. The system of claim 15, wherein the elastic bands further comprise a continuous radio opaque stripe.

21. The system of claim 15, wherein the elastic bands are comprised of reinforced silastic material.

22. The system of claim 15, wherein the elastic bands are of a length corresponding to the distance between the two anchor hooks that retain the elastic band.

23. The system of claim 15, wherein the elastic bands have a resistance to extension corresponding to a desired flexion capacity of the upper, lower, and intermediate vertebrae.

24. The system of claim 23, wherein the elastic bands have a thickness proportional to the resistance to extension.

25. The system of claim 23, wherein the resistance to extension of the elastic bands is defined by an internal variation in resistance.

26. The system of claim 23, wherein the elastic bands further comprise color coding representing the resistance to extension.

27. The system of claim 15, wherein the anchor hooks are comprised of titanium material.

28. The system of claim 15, wherein each anchor hook is of a size appropriate to the vertebra to which it is attached.

29. A method of applying a spinal stabilization system to a spine of a person comprising the steps of:
drilling two pilot holes in each vertebra to be flexibly connected;
applying an anchor hook to each pilot hole in the vertebrae to be flexibly connected;
applying elastic bands to the anchor hooks; and
crimping the anchor hooks to retain the elastic bands;

30. The method of claim 29, wherein the step of applying elastic bands to the anchor hooks further comprises the steps of:
using an elastic band application tool comprising two pivotable lever arms, an anchor hook interface, a locking mechanism, and an elastic band rolling mechanism to extend the band over the anchor hooks, wherein the anchor hook interface of the elastic band application tool mates with a groove on each of the anchor hooks to which an elastic band is applied; and
releasing the elastic band from the elastic band application tool onto the anchor hooks.
